(19) United States
(12) Patent Application Publication
(10) Pub. No.: US 2009/0048550 A1
(43) Pub. Date: Feb. 19, 2009

(54) KNEE PADS FOR CHILDREN AND METHOD OF MANUFACTURE

(75) Inventors: Stacey Feeley, Los Angeles, CA (US); Giulianna Schwab, Los Angeles, CA (US)

Correspondence Address: TSIRCOU INTELLECTUAL PROPERTY LAW, 655 Central Ave., 17th Floor, Glendale, CA, 91203, US

(73) Assignee: SILIKIDS LLC, Los Angeles, CA (US)

(21) Appl. No.: 11/840,967

(22) Filed: Aug. 18, 2007

(51) Int. Cl. A61F 5/00; A41D 13/06

(52) U.S. Cl. 602/26; 2/24

(57) ABSTRACT

A knee pads are provided comprising a fabric tube configured to cover a knee region of a child, preferably sized for infants and/or toddlers, and a silicone-based pad disposed over the patella of the child, when worn. The fabric tube includes elastic material disposed in an upper end and in a lower end of the tube to secure about the knee. The pad is molded onto the outer surface of the fabric tube. The fabric tube can include an upper cuff, a lower cuff, and a main region between the upper cuff and the lower cuff that circumscribes the knee region in a contiguous manner.

### FIELD OF THE INVENTION

The present invention relates generally to knee pads and, more particularly, to knee pads configured to protect children, including crawling infants.

### BACKGROUND OF THE INVENTION

Crawling is a critical part of an infant's development. It helps promote coordination, balance, and overall physical development. It also promotes mental development by exercising the infant's sense of curiosity and exploration. Accordingly, it is important to encourage infants to crawl often. Infants crawl on a variety of surfaces, including hard surfaces and surfaces that could irritate the skin. Thus, unless adequately protected, an infant's knees could scrape or become irritated as they crawl. Unfortunately, little attention has been paid to address this concern.

Infant apparel may include a certain measure of reinforcement over the knees. However, infant's clothing is typically loose fitting, so the reinforcement often slides out of position, minimizing its effectiveness. Moreover, it is inconvenient and impractical to dress an infant, at all times, prior to allowing the infant to crawl.

Others have attempted to provide knee pads for infants that address concerns with reinforced apparel. Although generally effective, shortfalls exist. For example, certain knee pad designs include fairly complicated strap systems that can be uncomfortable and difficult to use. Also, many knee pads include fabric covering the padded portions over the knees that contact the ground while crawling. Such knee pads can snag on rough surfaces.

It should, therefore, be appreciated that there remains a need for a knee pad that addresses the aforementioned shortcomings. The present invention fulfills this need and others.

### SUMMARY OF THE INVENTION

Briefly, and in general terms, the invention provides a knee pad for a child comprising a fabric tube configured to cover a knee region of a child and a silicone-based pad disposed over the patella of the child, when worn. The fabric tube includes elastic material disposed in an upper end and in a lower end of the tube to secure about the knee. The pad is molded onto the outer surface of the fabric tube.

More particularly, and in an exemplary embodiment, the knee pad includes a knit fabric tube configured to circumscribe a knee region of a child, preferably sized for infants and/or toddlers. The fabric tube includes an upper cuff, a lower cuff, and a main region between the upper cuff and the lower cuff that circumscribes the knee region in a contiguous manner. The upper cuff and the lower cuff have elastic material knit therein. The main region includes cotton yarn forming terry loops directed inwardly to be disposed against the child. The terry loops can be between about 1 mm and about 7 mm in length.

In a detailed aspect of an exemplary embodiment, the upper and the lower cuffs each include two layers of fabric formed by folding over a portion of the fabric inwardly and sewing the portion in place.

In another detailed aspect of an exemplary embodiment, an outer surface of the pad defines a plurality of grooves. The grooves can form a plurality of concentric, oval rings.

The invention also provides a method of manufacturing a knee pad for a child, comprising, forming a knit fabric tube configured to circumscribe a knee region of a child, the fabric tube having an upper cuff, a lower cuff, and a main region between the upper cuff and the lower cuff that circumscribes the knee region in a contiguous manner, the upper cuff and the lower cuff having elastic material knit therein, and the main region including cotton yarn forming terry loops directed inwardly to be disposed against the child; and molding silicone-based pad onto an outer surface of the knit fabric tube such that the pad is disposed over the patella of the child when worn.

For purposes of summarizing the invention and the advantages achieved over the prior art, certain advantages of the invention have been described herein. Of course, it is to be understood that not necessarily all such advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other advantages as may be taught or suggested herein. Moreover, a number of features are discussed throughout the specification with reference to particular embodiments. Nonetheless, those skilled in the art will appreciate that various other embodiments can be provided without departing from the scope of the invention, to include any and all combination of features discussed.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment disclosed.

### BRIEF DESCRIPTION OF THE DRAWINGS

Embodiments of the present invention will now be described, by way of example only, with reference to the following drawings in which:

FIG. 1 is a perspective view of a knee pad in accordance with the new design, as worn by an infant (shown in phantom).

FIG. 2. depicts a cross-sectional view of the knee pad of FIG. 1.

FIG. 3 is a front elevational view of the knee pad of FIG. 1.

FIG. 4 is a side elevational view of the knee pad of FIG. 1.

### DETAILED DESCRIPTION OF THE PREFERRED EMBODIMENTS

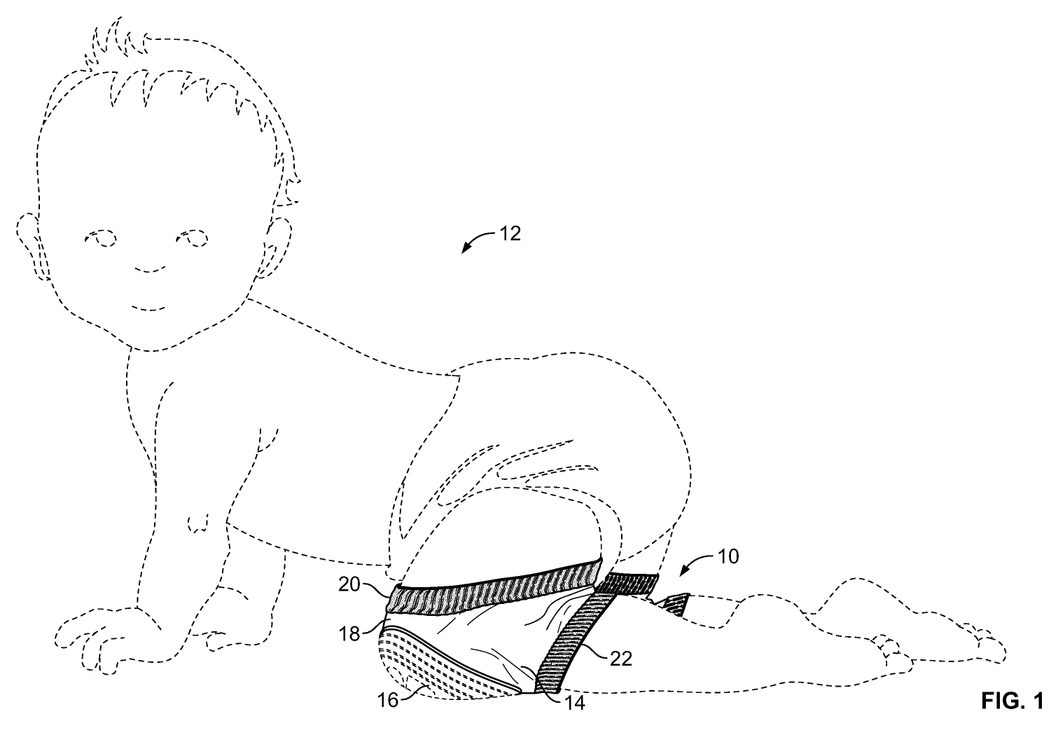

Referring now to the drawings, and particularly FIG. 1, there is shown a knee pad 10 as worn by an infant 12. The knee pad includes a knit fabric tube 14 and a pad 16 molded onto an outer surface 18 of the tube over the patella of the infant. The fabric tube includes upper and lower cuffs 20, 22 that have elastic material to aid in securing the knee pad on the infant, when worn. The fabric tube is formed of soft, plush material to ensure comfort for the infant. The pad is formed of resilient, durable material to protect the infant during use, while facilitating the useful life of the knee pad.

The fabric tube 14 is approximately 4 inches (10.16 cm) in length and 2.5 inches (6.35 cm) in width. The fabric tube 14 includes a main section 24 disposed between the upper and the lower cuffs 20, 22. The main section is about 3 inches (7.62 cm) in length and is generally tubular in shape such that it circumscribes the knee region of the infant in a contiguous manner. The main section is includes cotton yarn knit to form terry loops 26 directed inward, to provide a measure of cushioned support, promoting comfort for the infant. Moreover, the terry loops facilitate breathability of the knee pad, further promoting comfort.

In the exemplary embodiment, the main section 24 of the fabric tube 14 has a generally constant concentration of terry loops 26 throughout. Preferably, the terry loops are between about 0.5 mm up to about 20 mm in length, more preferably, between about 1 mm and about 7 mm in length. In the exemplary embodiment, the terry loops are about 3 mm in length.

Both the upper and the lower cuffs 20, 22 are knit in a continuous manner to opposing ends of the main section 24. Both cuffs are formed of a combination of cotton yarn and elastic thread knit together. The elastic thread is knit such that it circumscribes the leg of the infant. In the exemplary embodiment, the knit pattern of the cuffs differ from the knit pattern used on the main section. More particularly, the cuffs exclude terry loops as used in the main section.

The cuffs 20, 22 include two layers material formed by folding a portion of the cuff inward an sewing it in place with a stitch line 28, 30. For the lower cuff, the stitch line is space away a prescribed distance from an edge 34 causing a lower edge portion 38 to curl upon itself. In this manner, edge portions facilitate a grip that aids in securing the knee pad on the infant.

More particularly, the lower edge portion 38 is formed of a soft, lightweight cotton yarn knit into a comparatively thinly weighted material relative to other portions of the fabric tube 14. With the stitch line spaced away from the edge 34, the lower edge portion is inclined to curl upon itself. When so curled, the edge portion provide a plush grip against the infant's skin. In other embodiments, a curled edge portion can also be used on the upper cuff or just on the upper cuff or excluded entirely.

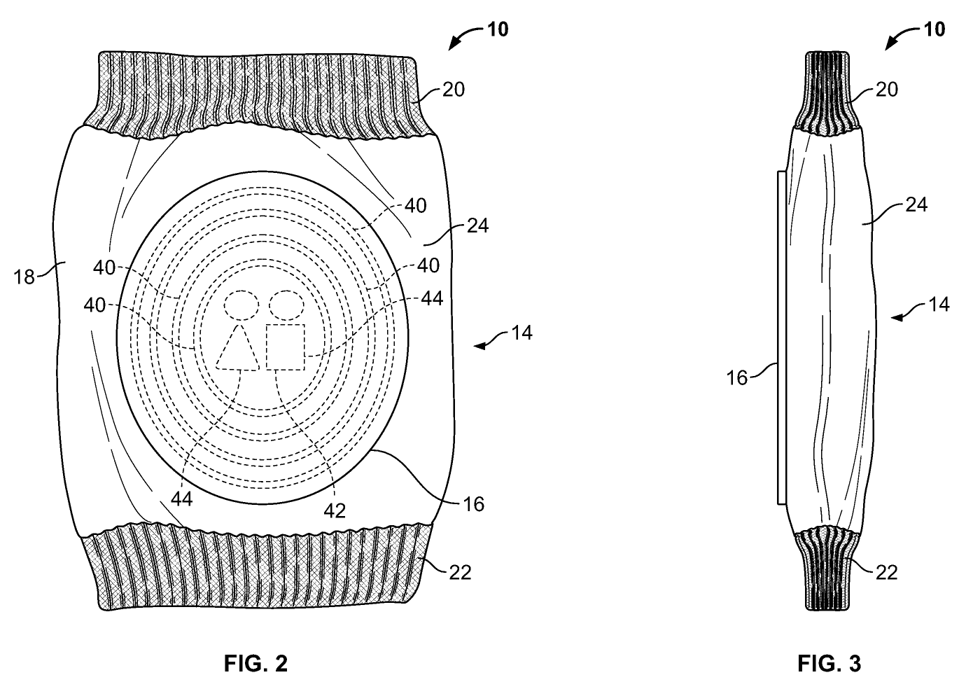

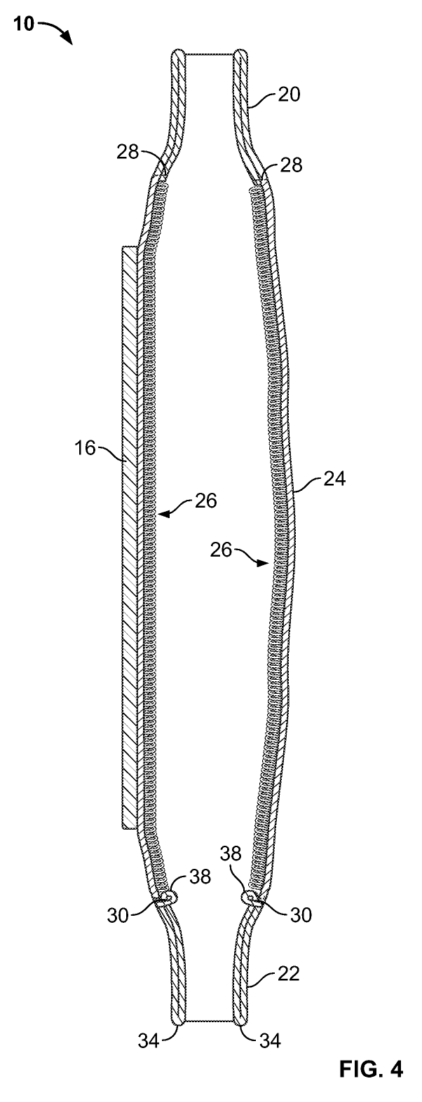

With reference to FIGS. 2 and 3, the pad 16 is molded onto the outer surface 18 of the fabric tube 12 positioned over the patella of an infant. The pad is made of durable, resilient material formed of a silicone-based material. More particularly, a silicon rubber (e.g., polysiloxanes) is used. In other embodiments, various other flexible material can be used, such as, thermoplastic elastomer (TPE), flexible PVC or the like. The pad is generally oval having a semi-major axis of about 2.25 inches (5.715 cm) and a semi-minor axis of about 2 inches (5.08 cm). Preferably, the thickness of the pad is between about 0.3 mm and about 20 mm, more preferably, between about 0.5 mm and about 10 mm. In the exemplary embodiment, a thickness of about 1 mm is used.

The pad 16 includes a plurality of grooves 40 defined by the outer surface of the pad. The grooves aid in providing a measure of traction for the infant while crawling. In the exemplary embodiment, the grooves are arranged as concentric rings, or ovals, disposed about a central region 42 of the pad. The central region includes central grooves 44 that also aid in providing traction. The central grooves also provide ornamentation, in that they outline a logo.

It should be appreciated from the foregoing that the present invention provides a knee pad having a fabric tube configured to cover a knee region of an infant and a silicone-based pad. the fabric tube has elastic material disposed in an upper end and in a lower end of the tube to secure about the knee. The silicone-based pad is molded onto an outer surface of the fabric tube such that the pad is disposed over the patella of the infant, when worn.

Although the invention has been disclosed in detail with reference only to the exemplary embodiments, those skilled in the art will appreciate that various other embodiments can be provided without departing from the scope of the invention, to include any and all combination of features discussed. Accordingly, the invention is defined only by the claims set forth below.

What is claimed is:

1. A knee pad for a child, comprising:
a fabric tube configured to cover a knee region of a child, the fabric tube having elastic material disposed in an upper end and in a lower end of the tube to secure about the knee, the fabric tube having an outer surface and an inner surface against the skin of the child; and
a silicone-based pad molded onto the outer surface of the fabric tube such that the pad is disposed over the patella of the child, when worn.

2. A knee pad as defined in claim 1, wherein the fabric tube contiguously circumscribes the knee region of the child, between the upper end and the lower end thereof.

3. A knee pad as defined in claim 1, wherein the upper end and the lower end of the fabric tube include elastic bands integrally knitted therein.

4. A knee pad as defined in claim 1, wherein the fabric tube includes a main portion having knitted cotton yarn forming terry loops.

5. A knee pad as defined in claim 4, wherein the terry loops are between about 1 mm and about 7 mm in length.

6. A knee pad as defined in claim 1, wherein the upper end and the lower end, each include two layers of fabric formed by folding over a portion of the fabric inwardly and sewing the portion in place.

7. A knee pad as defined in claim 1, wherein an outer surface of the pad defines a plurality of grooves.

8. A knee pad as defined in claim 7, wherein the plurality of grooves form a plurality of concentric rings.

9. A knee pad for a child, comprising:
a knit fabric tube configured to circumscribe a knee region of a child, the fabric tube having an upper cuff, a lower cuff, and a main region between the upper cuff and the lower cuff that circumscribes the knee region in a contiguous manner, the upper cuff and the lower cuff having elastic material knit therein, and the main region including cotton yarn forming terry loops directed inwardly to be disposed against the child; and
a silicone-based pad molded onto an outer surface of the knit fabric tube such that the pad is disposed over the patella of the child when worn.

10. A knee pad as defined in claim 9, wherein an outer surface of the silicone-based pad defines a plurality of grooves.

11. A knee pad as defined in claim 9, wherein the plurality of grooves form a plurality of concentric rings.

12. A knee pad as defined in claim 9, wherein the silicone-based pad is generally oval.

13. A knee pad as defined in claim 9, wherein in the main region of the fabric tube has a generally content concentration of terry loops throughout.

14. A knee pad as defined in claim 9, wherein the upper cuff and the lower cuff, each include two layers of fabric formed by folding over a portion of the fabric inwardly and sewing the portion in place.

15. A knee pad as defined in claim 14, wherein the upper cuff and the lower cuff lack terry loops.

16. A method of manufacturing a knee pad for a child, comprising:
forming a knit fabric tube configured to circumscribe a knee region of a child, the fabric tube having an upper cuff, a lower cuff, and a main region between the upper cuff and the lower cuff that circumscribes the knee region in a contiguous manner, the upper cuff and the lower cuff having elastic material knit therein, and the main region including cotton yarn forming terry loops directed inwardly to be disposed against the child; and
molding silicone-based pad onto an outer surface of the knit fabric tube such that the pad is disposed over the patella of the child when worn.

17. A method as defined in claim 16, wherein the forming step further comprises:
knitting a first tubular section having elastic material knit therein,
knitting a main tubular section to the first tubular section, the main tubular section having a generally constant concentration of terry loops that face inwardly,
knitting a second tubular section to the main tubular section on a side opposing the first tubular section, the second tubular section having elastic material knit therein,
forming a first cuff by folding portion of the first tubular section inward and sewing the portion in place, and
forming a second cuff by folding portion of the second tubular section inward and sewing the portion in place.

18. A method as defined in claim 16, wherein the forming step further includes forming upper cuff and the lower cuff lack terry loops.
